(19) United States
(12) Patent Application Publication
(10) Pub. No.: US 2024/0115624 A1
(43) Pub. Date: Apr. 11, 2024

(54) PHARMACEUTICAL COMPOSITION FOR TREATMENT AND PREVENTION OF INFLAMMATORY SKIN DISEASES COMPRISING RUMINOCOCCUS GNAVUS AS ACTIVE INGREDIENT

(71) Applicants: THE ASAN FOUNDATION, Seoul (KR); UNIVERSITY OF ULSAN FOUNDATION FOR INDUSTRY COOPERATION, Ulsan (KR)

(72) Inventor: Soo-Jong HONG, Seoul (KR)

(73) Assignees: THE ASAN FOUNDATION, Seoul (KR); UNIVERSITY OF ULSAN FOUNDATION FOR INDUSTRY COOPERATION, Ulsan (KR)

(21) Appl. No.: 18/482,063

(22) Filed: Oct. 6, 2023

(30) Foreign Application Priority Data
Oct. 7, 2022 (KR) 10-2022-0128306
Jan. 18, 2023 (KR) 10-2023-0007083

(51) Int. Cl. A61K 35/74; A61K 9/00; A61P 17/00

(52) U.S. Cl. CPC A61K 35/74; A61K 9/0056; A61P 17/00; A61K 2035/11

(57) ABSTRACT

A method for treating an inflammatory skin disease, including administering to a subject in need thereof a pharmaceutical composition containing Ruminococcus gnavus as an active ingredient. A method for treating an inflammatory skin disease, including applying to a subject in need thereof a cosmetic composition containing Ruminococcus gnavus as an active ingredient.

### CROSS-REFERENCE TO RELATED APPLICATION

This application claims the benefit of Korean Patent Application No. 10-2022-0128306 filed on Oct. 7, 2022, in the Korean Intellectual Property Office, the entire disclosure of which is incorporated herein by reference for all purposes.

This application claims the benefit of Korean Patent Application No. 10-2023-0007083 filed on Jan. 18, 2023, in the Korean Intellectual Property Office, the entire disclosure of which is incorporated herein by reference for all purposes.

### BACKGROUND

### 1. Field of the Invention

The present disclosure provides a pharmaceutical composition for prevention or treatment of inflammatory skin diseases including Ruminococcus gnavus as an active ingredient.

### 2. Description of the Related Art

The prevalence of atopic dermatitis worldwide is about 20%, of which about 80% occurs among preschool children with a continuous increase, but the cause of the increase is not yet known, while several hypotheses support the mechanism of onset. The immune system develops within 3 years after birth, and the composition of intestinal microbiome, which is closely related to immune development, is created in this stage, and it is reported that changes in the intestinal microbiome at this stage will have a decisive impact on the occurrence of atopic dermatitis.

Although causes of atopic dermatitis may vary due to complicated clinical symptoms appearing by the interaction of genetic and environmental factors, atopic dermatitis is mainly attributable to environmental factors, genetic predisposition, abnormalities in immunological responses and skin barriers, and family history, including environmental pollution such as smoke, increased uses of food additives, and an increase in substances (allergens) that cause allergies such as house dust mites due to elevated indoor temperature.

Many therapies have been used, such as uses of moisturizers for cleanness of skin and dryness prevention as a basic treatment for atopic dermatitis, uses of therapeutic agents such as oral antihistamines, steroids, immunosuppressants, antibiotics, antiviral agents, antifungal agents, and probiotics for skin inflammation relief, and photochemotherapy, but a long-term use of these preparations causes side effects such as skin atrophy and swelling, expansion of blood vessel and pores, and skin discoloration, with concerns on the disease and side effects of drugs such as steroids while atopic dermatitis becomes chronic or recurs frequently due to the nature of atopic dermatitis, such that there are many cases resulting in chronic diseases with repetition of exacerbation and alleviation due to improper treatment and reliance on unverified treatment methods.

Therefore, there is a steady demand for natural atopic dermatitis treatments with no side effects while ensuring safety in human body.

### PRIOR ART DOCUMENT

### Patent Document

(Patent Document 1) Japanese Patent Application Laid-Open Publication No. 32536865 (published on Dec. 17, 2020)

### SUMMARY

### Problem to be Solved by the Invention

Accordingly, an object of the present disclosure is to provide a pharmaceutical composition for prevention or treatment of inflammatory skin diseases including Ruminococcus gnavus as an active ingredient.

Another object of the present disclosure is to provide a health functional food composition for prevention or alleviation of inflammatory skin diseases including Ruminococcus gnavus as an active ingredient.

Another object of the present disclosure is to provide a cosmetic composition for prevention or alleviation of inflammatory skin diseases including Ruminococcus gnavus as an active ingredient.

### Means for Solving the Problem

To achieve the above object, the present disclosure provides a pharmaceutical composition for prevention or treatment of inflammatory skin diseases including Ruminococcus gnavus as an active ingredient.

In addition, the present disclosure provides a health functional food composition for prevention or alleviation of inflammatory skin diseases including Ruminococcus gnavus as an active ingredient.

In addition, the present disclosure provides a cosmetic composition for prevention or alleviation of inflammatory skin diseases including Ruminococcus gnavus as an active ingredient.

### Effects of the Invention

According to the present disclosure, it was found that Ruminococcus gnavus suppresses expression of IL-4, IL-8, and IL-13 and increases production of short chain fatty acids, and the use of bacteria may provide more safe and diverse applicability in pharmaceutical compositions, health functional food compositions, and cosmetic compositions for inflammatory skin diseases.

### BRIEF DESCRIPTION OF THE DRAWINGS

FIG. 1 shows results of identifying epidermal permeability barrier and skin histology in atopic dermatitis animal models caused by Ruminococcus gnavus

FIG. 2 shows results of identifying effects of Ruminococcus gnavus on systemic immune allergic reactions and the expression of Th2-related cytokine expression

FIG. 3 shows a result of identifying effects of Ruminococcus gnavus on IL-8 expression in human epithelial cells.

FIG. 4 shows results of identifying effects of Ruminococcus gnavus on CD4+Foxp3+ cells.

FIG. 5 shows results of identifying effects of Ruminococcus gnavus on short chain fatty acids in animal models.

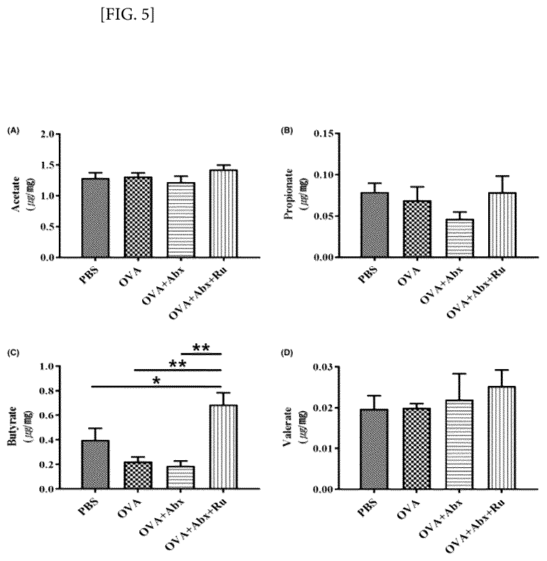

### DETAILED DESCRIPTION

Hereinafter, the present disclosure will be described in more detail.

The present disclosure provides a pharmaceutical composition for prevention or treatment of an inflammatory skin disease including Ruminococcus gnavus as an active ingredient.

The inflammatory skin disease may be any one selected from the group consisting of atopic dermatitis, allergic dermatitis, psoriasis, seborrheic dermatitis, contact dermatitis, erythematous lupus, and papular urticaria, but is not limited thereto.

The Ruminococcus gnavus may suppress expression of any one or more selected from the group consisting of IL-4, IL-8, and IL-13 or increase production of a short chain fatty acid, but is not limited thereto.

The short chain fatty acid may be any one or more selected from the group consisting of acetate, propionate, butyrate, and valerate, but is not limited thereto.

The pharmaceutical composition may be formulated in the form of an oral preparation, an injectable preparation, or a topical agent, but is not limited thereto.

In the pharmaceutical composition, Ruminococcus gnavus may be included in an amount of 0.01 to 10 parts by weight with respect to 100 parts by weight of the total composition.

In another example embodiment of the present disclosure, the pharmaceutical composition may further include one or more appropriate additives selected from the group consisting of carriers, excipients, disintegrants, sweeteners, coating agents, swelling agents, antifriction agents, lubricants, flavoring agents, antioxidants, buffers, bacteriostatic agents, diluents, dispersants, surfactants, binders, and lubricants that are commonly used in preparation of pharmaceutical compositions. Specifically, lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oil may be used as carriers, excipients, and diluents, and solid preparations for oral administration include tablets, pills, powder, granules and capsules, wherein such solid preparation may be prepared by mixing, in the composition, at least one or more excipients such as starch, calcium carbonate, sucrose or lactose, and gelatin. In addition to simple excipients, lubricants such as magnesium stearate and talc may also be used. Liquid preparations for oral use may include suspensions, solutions, emulsions, and syrups, and various excipients such as wetting agents, sweeteners, fragrances, and preservatives may be included in addition to commonly used simple diluents such as water and liquid paraffin. Preparations for parenteral administration include sterile aqueous solutions, non-aqueous solutions, suspensions, emulsions, freeze-dried preparations, and suppositories. Propylene glycol, polyethylene glycol, vegetable oils such as olive oil, and injectable esters such as ethyl oleate may be used as non-aqueous solvents and suspending agents. Witepsol, macrogol, Tween 61, cacao butter, laurin fat, and glycerogelatin may be used as a base of the suppositories. According to an example embodiment of the present disclosure, the pharmaceutical composition may be administered to a subject in a conventional manner via intravenous, intraarterial, intraperitoneal, intramuscular, intraarterial, intraperitoneal, intrasternal, transdermal, intranasal, inhalational, topical, rectal, oral, intraocular, or intradermal routes. The dosage of the active ingredient according to the present disclosure may vary depending on the condition and weight of the subject, the type and severity of disease, the drug form, the route of administration and the duration and be appropriately selected by those skilled in the art, and the daily dose may be 0.01 mg/kg to 200 mg/kg, preferably 0.1 mg/kg to 200 mg/kg, more preferably 0.1 mg/kg to 100 mg/kg. Administration may be conducted once a day or in several divided doses, but the scope of the present disclosure is not limited thereby.

In addition, the present disclosure provides a health functional food composition for prevention or alleviation of an inflammatory skin disease including Ruminococcus gnavus as an active ingredient.

The health functional food may include various nutritional supplements, vitamins, minerals (electrolytes), flavoring agents such as synthetic flavoring agents and natural flavoring agents, colorants and thickeners (cheese, chocolate, etc.), pectic acid and salts thereof, alginic acid and salts thereof, organic acids, protective colloidal thickeners, pH regulators, stabilizers, preservatives, glycerin, alcohol, and carbonating agents that are used in carbonated beverages. Additionally, it may also include pulp for manufacture of natural fruit juices, synthetic fruit juices, and vegetable beverages. These components may be used independently or in combination. In addition, the health functional food composition may be in any one form of meat, sausage, bread, chocolate, candy, snacks, confectionery, pizza, ramen, chewing gum, ice cream, soup, beverage, tea, functional water, drink, alcohol, and vitamin complex. In addition, the health functional food may further include food additives, and the suitability as the “food additive” is determined by the standards and criteria related to corresponding items according to the general rules and general test methods of Korean Food Additives Codex approved by the Ministry of Food and Drug Safety, unless otherwise stipulated. The items listed in the “Korean Food Additives Codex” may include, for example, chemically synthesized compounds such as ketones, glycine, potassium citrate, nicotinic acid, and cinnamic acid, natural additives such as persimmon color, licorice extracts, crystallized cellulose, kaoliang color, and guar gum, and mixed preparations such as sodium L-glutamate preparations, noodle-added alkali agents, preservative agents, and tar color agents. In this case, the content of active ingredients added to the food in the process of manufacturing the health functional food may be appropriately adjusted as needed, and preferably 1 to 90 parts by weight may be added to be included in 100 parts by weight of food.

In addition, the present disclosure provides a cosmetic composition for prevention or alleviation of an inflammatory skin disease including Ruminococcus gnavus as an active ingredient.

The cosmetic composition may have any one formulation selected from the group consisting of solutions, softening toners, nourishing toners, nourishing creams, moisturizing creams, nourishing essences, gels, and lotions, but is not limited thereto.

If the composition of the present disclosure is a cosmetic composition, the cosmetic composition may include conventional adjuvants such as stabilizers, solubilizers, vitamins, pigments, and fragrances and carriers in addition to the active ingredients. In addition, the cosmetic composition may additionally include a skin absorption promoter to enhance the effect.

The formulation of the cosmetic composition may be prepared in any formulation conventionally prepared in the art, wherein the cosmetic composition, for example, may be formulated as an emulsion, skin, toner, sunscreen, makeup base, foundation, powder, pack, ointment, patch, stick, shampoo, conditioner, spray, makeup remover, and cleanser, but is not limited thereto.

If the formulation is a paste, cream, or gel, animal oil, vegetable oil, wax, paraffin, starch, tracanth, cellulose derivatives, polyethylene glycol, silicone, bentonite, silica, talc, or zinc oxide may be used as a carrier component.

If the formulation is powder or a spray, lactose, talc, silica, aluminum hydroxide, calcium silicate or polyamide powder may be used as a carrier component, and in particular, in the case of the spray, chlorofluorohydrocarbon, propane/butane or a booster such as dimethyl ether may be additionally included.

If the formulation is a solution or emulsion, solvents, solubilizers or emulsifiers are used as a carrier component, for example, water, ethanol, isopropanol, ethyl carbonate, ethyl acetate, benzyl alcohol, benzyl benzoate, propylene glycol, 1,3-butylglycol oil, glycerol fatty ester, polyethylene glycol, or fatty acid ester of sorbitan.

If the formulation is a suspension, liquid diluents such as water, ethanol, or propylene glycol, a suspending agent such as ethoxylated isostearyl alcohol, polyoxyethylene sorbitol ester, and polyoxyethylene sorbitan ester, microcrystalline cellulose, aluminum metahydroxide, bentonite, agar, or tracanth may be used as the carrier component.

Hereinafter, example embodiments will be described in detail to help the understanding of the present disclosure. However, the following example embodiments are merely illustrative of the content of the present disclosure, and the scope of the present disclosure is not limited to the following examples. The example embodiments of the present disclosure are provided to more completely explain the present disclosure to those of ordinary skill in the art.

### <Experimental Example 1> Preparation of an Animal Model

All experimental procedures on mice were reviewed and approved by the Animal Care and Use Committee of Asan Medical Center (Authorization No. 2019-12-296), and all studies were conducted in accordance with the guidelines and regulations set by the veterinary hospital. Female BALB/c mice (4 weeks old, n=5/group) were purchased from Orient Bio (Seongnam, South Korea). To induce an imbalance of intestinal flora, mice were exposed to an antibiotics drinking water (1 g/L ampicillin, 500 mg/L vancomycin hydrochloride, 1 g/L neomycin (Sigma Aldrich, St Louis, MO), and 1 g/L metronidazole (Fresenius, Germany)). Prior to primary sensitization, mice were orally administered with Ruminococcus gnavus or phosphate buffered saline (PBS; pH 7.4) for 2 weeks. After administration of antibiotics for 2 weeks, the atopic dermatitis occurred with skin sensitization of ovalbumin (OVA; Ovalbumin; grade V; Sigma, St Louis, MO). The 6-week-old mice were anesthetized, whose backs were then shaved and subjected to induction of repeated skin damage by tape removal. Subsequently, 100 μl of PBS or 100 μg of OVA (grade V; Sigma) prepared by solely using PBS was coated on a sterile patch, and then an adhesive tape was used to be attached to an area where the tape was peeled off. OVA treatment was repeated once during 1 week of sensitization period, followed by a rest period of 2 weeks. After that, second sensitization period was followed for 1 week. Afterwards, the mice were euthanized, and samples were collected after 24 hours.

Ruminococcus gnavus (ATCC 29149) cultured in tryptic soy broth media (TSB, Oxoid, UK) containing 0.5% mucin was cultured overnight under anaerobic conditions at 37° C. The bacteria cultured in the optical density of (OD)₆₀₀=1 were then washed once with sterile PBS and resuspended in 200 μl of sterile PBS.

### <Experimental Example 2> Assessment of Transepidermal Water Loss and Clinical Scores and Histopathological Analysis of Skin Lesions

Assessment of transepidermal water loss (hereinafter referred to as TEWL) was measured via VapoMeter (SWL-3; Delfin Technologies). Clinical scores for skin lesions in mice were scored for erythema, keratin, and edema using a 0-3 scoring system: 0 (asymptomatic), 1 (mild), 2 (moderate), and 3 (severe). TEWL and clinical score values were assessed at baseline and after sensitization periods, respectively.

The change in epidermal thickness and the degree of inflammatory cells infiltration in the dermal layer were confirmed through histopathological analysis through staining with hematoxylin and eosin (H&E).

### <Experimental Example 3> Identification of Serum Total IgE and Cytokine Levels

Total IgE and OVA-specific IgE levels in serum were measured using enzymatic immunoassay (ELISA; eBioscience, San Diego, CA, USA). Th2-related cytokines (thymic stromal lymphopoietin (TSLP), interleukin (IL)-4, IL-6, and IL-13) and Treg-related cytokines (IL-10 and forkhead box P3 (Foxp3)) were measured using qPCR in skin tissues. Each signal was normalized for the GAPDH signal of the same sample.

In addition, to check the level of IL-8, 1.0×10⁵ cells/dish of Caco-2 cells were placed onto 60π cell culture dish and cultured at a confluency of 70˜80% in Dulbecco's modified Eagle's minimal essential medium (DMEM) supplemented with 10% fetal bovine serum (FBS) and 1× penicillin-streptomycin, and then the level of IL-8 was measured using qPCR.

### <Experimental Example 4> Flow Cytometry

Mouse mesenteric lymph nodes (MLN) and cutaneous draining lymph nodes were fixed and permeated using intracellular fixation and permeation buffer sets (eBioscience, San Diego, CA, USA), and investigation was followed using FITC-labeled anti-mouse CD4+, PE-labeled anti-mouse FOXP3+, and isotype control (all provided by eBioscience).

### <Experimental Example 5> Measurement of Short Chain Fatty Acid (SCFA) in Appendix Samples

Mouse cecum was collected, and 20 mg of cecal contents were suspended in the extract. SCFA was analyzed using a 7890A GC (Agilent Technologies) system and a 7000 GC/MS Triple Quad (Agilent Technologies). Extracted ion chromatograms corresponding to specific SCFAs were used for quantification.

### <Experimental Example 6> Statistical Analysis

A nonparametric test was used to compare the two groups (Mann Whitney U test). ANOVA and Newman-Keuls multiple comparative tests were used to assess the difference in the measurement among various groups. GraphPad Prism 4.0 (San Diego, CA) was used to perform statistical analysis. The independent sample t-test was performed using SPSS version 24.0 (SPSS Inc.).

### <Example 1> Identification of Skin Changes in Atopic Dermatitis Animal Models Caused by Ruminococcus Gnavus

In order to investigate whether Ruminococcus gnavus has the potential to alleviate atopic dermatitis, the effect of Ruminococcus gnavus on the epidermal permeable barriers and skin tissues was identified by comparing TEWL, clinical score, epidermal thickness and inflammatory cell infiltration in skin tissue in an atopic dermatitis induced animal model.

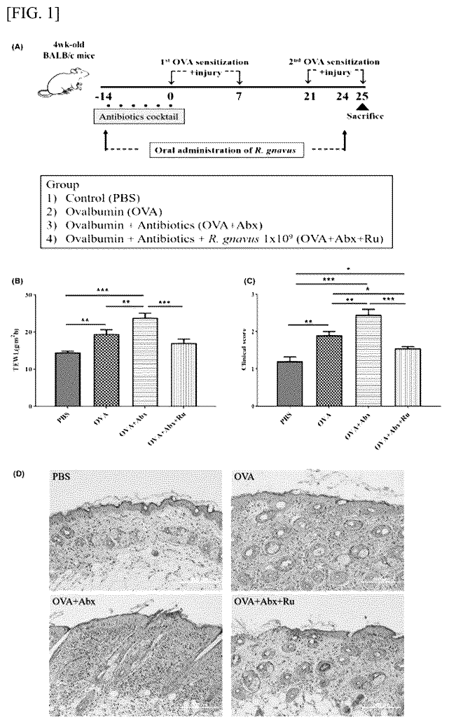

As a result, according to FIG. 1, TEWL and clinical scores of mice with atopic dermatitis developed by antibiotic treatment increased significantly, showing significant inflammatory cell infiltration and erythematous skin lesions compared to a control group exposed to OVA. However, the group of mice administered with Ruminococcus gnavus significantly reduced TEWL and clinical scores, as well as the epidermal thickness and skin infiltration of inflammatory cells.

This indicates that Ruminococcus gnavus effectively alleviated atopy-like symptoms in antibiotic-treated atopic dermatitis.

### <Example 2> Effects of Ruminococcus Gnavus on Systemic Immune Allergic Reactions and Cytokine Expression

To investigate the alleviation effect of Ruminococcus gnavus on serum IgE levels and expression of Th2-related cytokines in the skin, the inventors compared total serum IgE levels in mice orally administered with Ruminococcus gnavus compared to those with atopic dermatitis developed group that was treated with antibiotics.

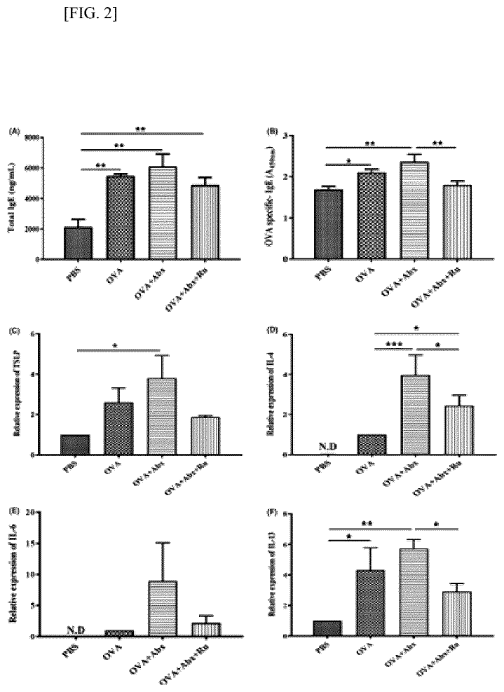

As a result, according to FIG. 2, it was found that total serum IgE levels were relatively reduced in mice orally administered with Ruminococcus gnavus. In addition, it was also found that administration of Ruminococcus gnavus to antibiotic-treated atopic dermatitis developed groups significantly inhibited OVA-specific IgE levels in the serum.

In addition, expression of Th2-related cytokines TSLP, IL-4, IL-6, and IL-13 in skin was measured. It was found that administration of Ruminococcus gnavus to antibiotic-treated atopic dermatitis developed groups significantly suppressed IL-4 and IL-13 expression.

This indicates that administration of Ruminococcus gnavus suppresses systemic allergy and Th2-related immune responses.

### <Example 3> Checking of Whether Ruminococcus Gnavus Regulates IL-8

To determine whether Ruminococcus gnavus regulates IL-8, Caco-2 cells were treated with TNF-α to induce inflammation and then treated with Ruminococcus gnavus.

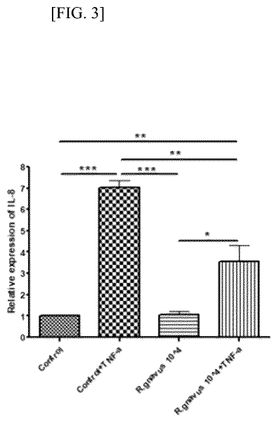

As a result, according to FIG. 3, it was found that IL-8 was significantly reduced, compared to a group treated with TNF-α, in the group treated with Ruminococcus gnavus in addition to the TNF-α.

### <Example 4> Checking of Whether Ruminococcus Gnavus Promotes Treg Responses and Regulates SCFA Levels

In the intestines of antibiotic-treated atopic dermatitis mice, the intestinal immune system regulates Treg and intestinal metabolites levels, contributing to immune tolerance. Investigation was conducted on whether administration of Ruminococcus gnavus affected the immunoregulation of intestinal and cutaneous draining lymph nodes.

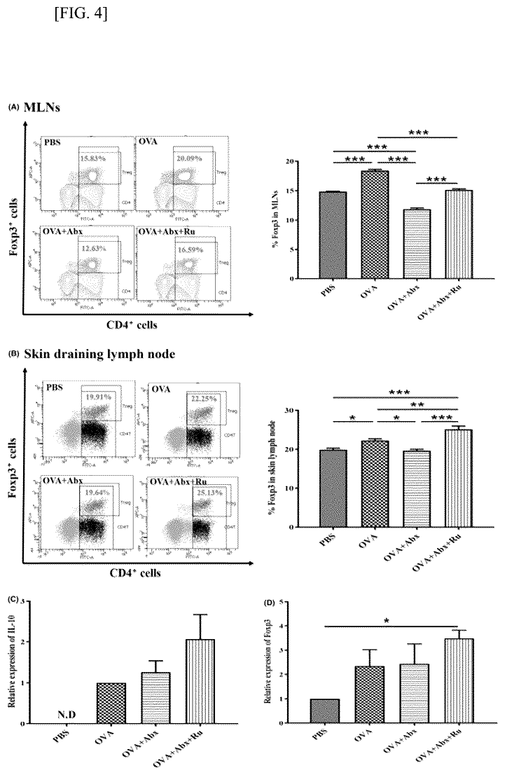

As a result, according to FIG. 4, it was found that administration of Ruminococcus gnavus to the atopic dermatitis developed group significantly increased CD4+FOXP3+ Treg population in intestinal and cutaneous draining lymph nodes. In addition, it was also observed that IL-10 and Foxp3 mRNA expression in the skin was increased by administration of Ruminococcus gnavus.

In addition, investigation was conducted on whether oral administration of Ruminococcus gnavus affected the level of intestinal microbiota metabolites. It was found that a concentration of S CFA significantly increased butyrate in the appendix when Ruminococcus gnavus was administered to the antibiotic-treated atopic dermatitis developed group.

This indicates that administration of Ruminococcus gnavus may suppress Th2-dominant immune responses in atopic dermatitis by enhancing butyrate production and inducing Treg-related responses.

The foregoing description of the present disclosure is for illustrative purposes only, and a person skilled in the art to which the present disclosure pertains will be able to understand that it may be easily modified into other concrete forms without altering the technical idea or essential features of the present disclosure. Therefore, the example embodiments described above should be understood as exemplary and not limited in all respects.

The scope of the present disclosure is indicated by the claims to be described below, and the meaning and scope of the claims and all changes or modified forms derived from the equivalent concept thereof should be construed as being included in the scope of the present disclosure.

1. A method for treating an inflammatory skin disease, comprising administering to a subject in need thereof a pharmaceutical composition comprising Ruminococcus gnavus as an active ingredient.

2. The method of claim 1, wherein the inflammatory skin disease is any one selected from the group consisting of atopic dermatitis, allergic dermatitis, psoriasis, seborrheic dermatitis, contact dermatitis, erythematous lupus, and papular urticaria.

3. The method of claim 1, wherein the Ruminococcus gnavus suppresses expression of any one or more selected from the group consisting of IL-4, IL-8, and IL-13 or increases production of a short chain fatty acid.

4. The method of claim 3, wherein the short chain fatty acid is any one or more selected from the group consisting of acetate, propionate, butyrate, and valerate.

5. The method of claim 1, wherein the pharmaceutical composition is formulated in a form of an oral preparation, an injectable preparation, or a topical agent.

6. The method of claim 1, wherein, in the pharmaceutical composition, Ruminococcus gnavus is included in an amount of 0.01 to 10 parts by weight with respect to 100 parts by weight of the total pharmaceutical composition.

7. A method for treating an inflammatory skin disease, comprising administering to a subject in need thereof a health functional food composition comprising Ruminococcus gnavus as an active ingredient.

8. A method for treating an inflammatory skin disease, comprising applying to a subject in need thereof a cosmetic composition comprising Ruminococcus gnavus as an active ingredient.

9. The method of claim 8, wherein the cosmetic composition has any one formulation selected from the group consisting of solutions, softening toners, nourishing toners, nourishing creams, moisturizing creams, nourishing essences, gels, and lotions.
